(19) United States
(12) Patent Application Publication
(10) Pub. No.: US 2004/0214794 A1
(43) Pub. Date: Oct. 28, 2004

(54) Use of lps for treating intestinal inflammation processes

(76) Inventors: Hans-Dieter Grimmecke, Feldschloschen (DE); Markus Bruno Kalousek, Schenkon (CH); Frank Seibold, Kehrsatz (DE)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 10/333,833

(22) PCT Filed: May 17, 2002
(86) PCT No.: PCT/EP02/05450

(30) Foreign Application Priority Data
May 23, 2001 (DE) 10125179.3

(51) Int. Cl. A61K 31/739

(52) U.S. Cl. 514/54

(57) ABSTRACT

The invention relates the use of LPS and derivarised variants thereof, especially E. coli, for the peroral or rectal treatment of intestinal inflammation processes and symptoms connected thereto.

The invention concerns the use of LPS in the treatment of intestinal inflammatory processes. LPS will be understood to mean naturally occurring and synthetically or semisynthetically prepared variants.

Lipopolysaccharides (LPS) consist of a lipid component, lipid A, and a polysaccharide unit covalently linked in this membrane domain. The polysaccharide region consists of the terminal O-specific chain, a substructure that comprises up to 50 repeating oligosaccharide units of usually two to eight monomers, and the core region, which is linked to the lipid A.

### O-specific chain—core region—lipid A

The O-specific chain is characterized by extreme structural variability in different species. The lipid A component is responsible for the biological activity described below. The biological activity is modulated by variation of the acylation pattern of the lipid A. This activity ranges from an agonistic effect, such as occurs with most naturally occurring LPS variants (e.g., Salmonella friedenau or Salmonella abortus equi), to the antagonistic effect of the LPS variants of plant-symbiotic microorganisms or synthetic derivatives (C. Alexander and E. T. Rietschel, Biospektrum, Vol. 4, No. 5, pp. 275-281, 1999,). A comprehensive description of LPS may also be found in A. Wiese, K. Brandenburg, U. Seydel, and S. Muller-Leoennies: The Dual Role of Lipopolysaccharide as Effector and Target Molecule. Biol. Chem., 380, pp. 767-784.

Lipopolysaccharides are formed by gram-negative bacteria and are highly potent stimulators of innate immunity. They bind to TLR4-/CD14 receptors on human mononuclear cells and induce the formation and secretion of

proinflammatory cytokines, such as TNFα, MIF, IL-1β, IL-6, IL-8, IL-12, IL-15 and IL-18,

various colony-stimulating factors,

various lipid mediators, and

reduced oxygen species.

In addition, LPS produces rapid-onset, antibody-dependent activation of the complement cascade with release of the anaphylatoxins C5a and C3a. Human T lymphocytes are stimulated to proliferate and to produce IL-2 and IFN (Alexander and Rietschel, 1999). During the further course of the inflammatory reaction, the formation of acute phase proteins is induced.

Lipopolysaccharides activate the nonspecific immune response by these mechanisms, and microorganisms, viruses and tumor cells can be better eliminated as a result of this response. Phenomena such as inflammation, fever and sepsis are induced by the LPS-induced production of cytokines and lipid mediators. Due to the toxic effect of LPS in sepsis, agonistic LPS are also called endotoxins.

The use of LPS antagonists in the treatment of sepsis and the use of LPS agonists to enhance immunological tumor eradication are the subjects of a variety of published papers (Grimminger et al., Internist 38, 541-552; Ikawa et al., J. Natl. Cancer Inst., 14, 1,195-1,201, 1954).

Inflammation can be the result of tissue damage following mechanical or chemical irritation, a reaction to infections, or the result of abnormal immunological reactions (autoimmune diseases, chronic inflammatory diseases). Acute inflammation is halted by elimination of the provoking factor, i.e., lysed cells in the case of tissue damage or pathogens in the case of infections. Chronic inflammation occurs when the provoking event cannot be eliminated, or if the immune response becomes independent, i.e., an autoimmune disease develops, in which the immune response is directed against autoantigens or against the ubiquitous intestinal flora.

The central event of all inflammatory reactions is leukocytic migration into the inflammatory focus. The recruited leukocytes release proinflammatory substances, substances that increase permeability, and toxic substances or trigger the release of such substances by other cells (cytokines, enzymes, mediators, prostaglandins, oxygen radicals). This leads to increased perfusion and to the activation and increased output of immunocytes, which are involved in the elimination of the pathogen and removal of decomposition products. Various growth factors, including cytokines, promote the proliferation of tissue cells and thus the healing process.

However, healthy cells are also attacked in the course of the inflammatory process. This leads to tissue damage, which is progressive in the case of chronic inflammation and results in tissue degeneration [Gemsa and Resch, 1997]. Inflammation of the intestinal mucosa also allows allergens and food antigens to penetrate the intestinal wall. The result is an excessive immune response to these epitopes. T lymphocytes, which are activated in the process, circulate in the lymphatic system and return to mucosal or dermal regions [Kantele et al., 1986], [Czerkinsky et al., 1987], [Wenneras et al., 1994], [Holmgren et al., 1989], [Sztein et al., 1994], [Kantele et al., 1999]. When they encounter the same or cross-reacting epitopes in these regions, this can provoke local or systemic allergic reactions, such as food allergy with urticaria, asthma, allergic rhinitis or neurodermatitis. There is thus a direct relationship among intestinal inflammation, increased permeability and allergic diseases [Hollander, 1999], [Ma, 1997], [Fink, 2001], [Nekam, 1998a], [Nekam, 1998b], [Pena, 1998], [Salminen et al., 1996], [Majamaa and Isolauri, 1997], [Dupont et al., 1989], [Malin et al., 1996]. See the list of references for details. For this reason, when intestinal inflammatory reactions are involved in the pathogenesis of allergic diseases, the elimination of intestinal inflammation is extremely important to causal treatment.

In infections with gram-negative bacteria, lipopolysaccharides are an important factor in the provocation of the acute inflammation. It has always been assumed, therefore, that LPS would have to intensify inflammatory processes.

Very surprisingly, it has now been found that LPS, either extracted from microorganisms or prepared synthetically or semisynthetically, are outstanding agents for the treatment of intestinal inflammatory processes.

LPS can be extracted from all gram-negative microorganisms and can be synthetically derived.

The effective dose of the substance is in the range of 0.05 ng/kg/d to about 100 μg/kg/d, and the preferred oral dosage range is about 1 ng/kg/d to 10 μg/kg/d. Since orally administered LPS is characterized by low toxicity, in strong contrast to parenterally administered LPS, a large therapeutic range is possible. Accordingly, no side effects have ever been observed, even after oral administration of relatively large doses.

LPS is preferably administered orally or rectally, e.g., in the form of solutions, powders, granules, tablets, capsules, coated tablets, suppositories or enemas. LPS can also be brought to the mucosal site of action by oral administration of LPS-producing or LPS-overproducing, nonpathogenic intestinal bacteria. However, transdermal, topical or parenteral administration is also possible, although the latter route has a much smaller therapeutic range.

When LPS is administered as a solution, it makes sense to select as the base an aqueous solvent similar to peptone with amphophilic, water-soluble substances, such as fatty acid derivatives and lipid derivatives, since particularly water-soluble substances with a lipophilic component provide good micelle formation as a condition of solubility. The same consideration should be given to solid forms of administration to ensure sufficient solubilization and bioavailability in the intestinal mucosa. The pharmaceutical form of administration is produced by standard methods with which the expert is familiar.

The LPS that is used is an agonistic LPS from Escherichia coli, preferably the strain Laves 1931, which shows a level of activity with respect to stimulation of TNFα synthesis in monocytes comparable to that of the reference LPS of Salmonella friedenau. The exact mechanism of the anti-inflammatory action is not yet known.

Since, however, as the literature describes (Alexander et al., Wiese et al.), the pharmacologic effect of LPS depends on its lipid A component and especially its acylation, and not on the O-specific chain or the core polysaccharide, it may be assumed that all LPS with an identical or similar lipid A acylation pattern and corresponding agonistic pharmacologic effect are similarly effective.

Orally administered LPS has a strong anti-inflammatory effect.

In a study on mice that develop severe intestinal inflammation due to an immunological defect (IL-10-/-knockout) [T. T. MacDonald, 1994], LPS suppressed inflammation, as was demonstrated both clinically and histologically.

In studies on mice with an intestinal inflammatory reaction due to a chemical noxa, LPS suppressed the inflammatory reaction, as could likewise be demonstrated both clinically and histologically.

The invention will be explained in greater detail by the following examples:

### EXAMPLE 1

### Preparative Isolation of LPS

LPS can be extracted from gram-negative bacteria by various methods. A preferred method is described here:

To produce the active LPS-containing fraction from E. coli, preferably E. coli of the serotype O2:K1:H6 (preferably the strain Laves 1931) is cultivated for five days at 37° C. to a growth density of 3×10⁸ colony-forming units/mL on a culture medium based on meat peptone. However, other culture media, other gram-negative microorganisms, and other growth densities may be used. A protein-free and cell-free extract is recovered from the cultivated culture by the standard operations of thermal lysis and multiple filtration. The extract is concentrated, dialyzed against water with a 1-kD membrane and lyophilized. Alternatively, the thermolysed biomass may be centrifuged at 3,900 g for 20 minutes, followed by lyophilization of the supernatant. After thorough dialysis against water with a 1-kD membrane, the retained material [=retentate] is concentrated and lyophilized.

The above method of isolating lipopolysaccharides is the method given by Westphal et al. [Westphal et al., 1952]. The lyophilized material [=lyophilizate] is suspended in a water/phenol mixture (55/45%) and homogenized. The phases are separated by centrifugation at 2,000 g.

The aqueous phase is dialyzed against water as described above, concentrated, and lyophilized.

The lyophilized material is dissolved in water in a concentration of 10-40 mg/mL and ultracentrifuged three times at 55,000 g for 22 hours. LPS-L31 is obtained by lyophilization of the sediment and may be further purified (e.g., by gel filtration).

### EXAMPLE 2

### Suppression of Intestinal Inflammatory Reaction in Autoimmune Diseases

The following experiments were conducted under double-blind conditions on interleukin-10-/-knockout mice [T. T. MacDonald, 1994]. Over a period of 21 days, two groups of five 4-week-old mice were given either placebo (bovine peptone) to drink or placebo +14 ng/mL of lipopolysaccharide from lysed Escherichia coli, strain Laves 1931 (LPS-L31, Treated). The weight development of the mice was monitored in the course of the treatment. Weight gain was significantly greater in the treated group than in the placebo group. Moreover, clinical signs of colitis, such as anal rubor, soft stool, and transanal discharge of blood, were more frequently observed in the placebo group.

After three weeks of treatment, the intestine was preserved, and the crypt index was determined. The crypt index is a measure of the degeneration or hyperpiasia of the intestinal wall and thus a measure of inflammation: the greater the value, the greater the inflammation. The treated mice had a crypt index of 4.75, whereas the placebo mice had a crypt index of 8.0, from which it can be concluded that the mice treated with LPS-L31 had significantly less inflammation than the placebo mice. The IL-10-/-knockout mouse is a recognized model of chronic inflammatory intestinal diseases in man.

The results show that LPS-L31 reduces inflammation due to abnormal immunologic regulation (autoimmune diseases).

### EXAMPLE 3

### Suppression of Intestinal Inflammatory Reactions on Exposure to a Chemical Noxa

In another double-blind experiment, two groups of six Balb-c mice (2 months old) were given placebo (bovine peptone) to drink or placebo +14 ng/mL LPS-L31 (treated) for one week. For five days after this one-week treatment period, all of the mice were given 2.5% DSS as a chemical noxa to produce inflammation. This was followed by another treatment with placebo or placebo plus treatment for six days. Weight gain was significantly greater in the treated group than in the placebo group. The crypt index was significantly lower in the treated group than in the placebo group, i.e., LPS-L31 reduced the intestinal inflammation. Moreover, clinical signs of colitis, such as anal rubor, soft stool, and transanal discharge of blood, were more frequently observed in the placebo group.

### EXAMPLE 4

### Determination of the Effect of LPS on the Formation of TNFα

To study the stimulation of TNFα secretion in mononuclear cells, we followed the procedure given in [Thomsen and Loppnow, 1995]: Mononuclear cells are isolated from the blood of healthy donors by density gradient ultracentrifugation with Ficoll®, washed, and incubated in culture medium (RPMI 1640). The cells are incubated for 24 hours with LPS or control solution, harvested, treated with 2% fetal calf serum, and stored at −20° C. until cytokine analysis. The TNFα is determined with a commercial ELISA (Enzyme-Linked Immunosorbent Assay) using anti-TNFα antibodies. 24 hours after addition of 1 μg/mL LPS-L31, a TNFα concentration of 1,851 pg/mL was measured. 1 μg/mL of reference LPS from Salmonella friedenau induced the production of 1,976 pg/mL TNFα in the same period of time. It follows that LPS-L31 is an agonistic LPS that does not differ in activity from known forms of LPS. Therefore, it can be expected that all agonistic forms of LPS are effective in intestinal inflammation. In particular, contrary to what one would expect on the basis of the previous literature (Alexander et al., Wiese et al.), no antagonistic effect is necessary to ensure efficacy.

### LIST OF REFERENCES

Alexander, C., Rietschel, E. T. (1999): Bacterial lipopolysaccharides—highly active stimulators of innate immunity. Biospectrum 5: 275-281.

Czerkinsky, C., Prince, S. J., Michalek, S. M., Jackson, S., Russell, M. W., Moldoveanu, Z., McGhee, J. R., Mestecky, J. (1987): IgA antibody-producing cells in peripheral blood after antigen ingestion: evidence for a common mucosal immune system in humans. Proc Natl. Acad. Sci. USA 84: 2,449-2,453.

Dupont, C., Barai, E., Molkhou, P., Raynaud, F., Barbet, J. P., Dehennin, L. (1989): Food-induced alterations of intestinal permeability in children with cow's milk-sensitive enteropathy and atopic dermatitis. J. Pediatr. Gastroenterol. Nutr. 8: 459-465.

Fink, M. P. (2001): Leaky gut hypothesis: a historical perspective (editorial). Cri.t Care Med. 18: 579-580.

Gemsa, D., Resch, K. (1997): Inflammation. In: Immunologie [Immunology], Gemsa, D., Kalden J. R., Resche, K. (eds.), pp. 135-158. Georg Thieme Verlag, Stuttgart.

Grimminger, F., Mayer, K., Seeger, W. (1997): Is there a guaranteed immunotherapy in sepsis? Internist 38: 541-552.

Hollander, D. (1999): Intestinal permeability, leaky gut, and intestinal disorders. Curr. Gastroenterol. Rep. 1: 410-416

Holmgren, J., Fryklund, J., Larsson, H. (1989): Gamma-interferon-mediated down-regulation of electrolyte secretion by intestinal epithelial cells: a local immune mechanism? Scand. J. Immunol. 30: 499-503.

Ikawa, M., Koepfli, J. B., Mudd, S. G., Niemann, C. (1954): An agent from E. coli causing hemorrhage and regression of an experimental mouse tumor. J. Natl. Cancer Inst. 14: 1,195-1,201.

Kantele, A., Arvilommi, H., Jokinen, I. (1986): Specific immunoglobulin-secreting human blood cells after peroral vaccination against Salmonella typhi. J. Infect. Dis. 153: 1,126-1,131.

Kantele, A., Zivny, J., Hakkinen, M., Elson, C. O., Mestecky, J. (1999): Differential homing commitments of antigen-specific T cells after oral or parenteral immunization in humans. J. Immunol. 162: 5,173-5,177.

Ma, T. Y. (1997): Intestinal epithelial barrier dysfunction in Crohn's disease (44099). P. S. E. B. M. 214: 318-327.

MacDonald, T. T. (1994): Gastrointestinal inflammation. Inflammatory bowel disease in knockout mice. Curr. Biol. 4: 261-263.

Majamaa, H., Isolauri, E. (1997): Probiotics: a novel approach in the management of food allergy. J. Allergy Clin. Immunol. 99: 179-185.

Malin, M., Isolauri, E., Pikkarainen, P., Karikoski, R., Isolauri, J. (1996): Enhanced absorption of macromolecules—A secondary factor in Crohn's disease. Dig. Dis. Sci. 41: 1,423-1,428.

Nekam, K. (1998a): Management of food allergy. Allergy 53: 122-124.

Nekam, K. L. (1998b): Nutritional triggers in asthma. Acta Microbiol. Immunol. Hung. 45: 113-117.

Pena, A. S. (1998): Food allergy, coeliac disease, and chronic inflammatory bowel disease in man. Vet. Q. 20: 49-52.

Salminen, S., Isolauri, E., Salminen, E. (1996): Clinical uses of probiotics for stabilizing the gut mucosal barrier: Successful strains and future challenges. Int. J. Microbiol. 70: 347-358.

Sztein, M. B., Wassermann, S. S., Tacket, C. O., Edelman, R., Hone, D., Lindberg, A. A., Levine, M. M. (1994): Cytokine production patterns and lymphoproliferative responses in volunteers orally immunized with attenuated vaccine strains of Salmonella typhi. J. Infect. Dis. 170: 1,508-1,517.

Thomsen, A., Loppnow, H. (1995): Cytokine production by mononuclear cells following stimulation with a peptide-containing, endotoxin-free Escherichia coli extract. Arzneim-Forsch/Drug Res. 45: 657-661.

Wenneras, C., Svennerholm, A. M., Czerkinsky, C. (1994): Vaccine-specific T cells in human peripheral blood after oral immunization with an inactivated enterotoxigenic Escherichia coli vaccine. Infect. Immunol. 62: 874-879.

Westphal, O., Luderitz, O., Bister, F., (1952): The extraction of bacteria with phenol/water. Z. Naturforsch., B: Chem. Sci. 7: 148-155.

1-10. (canceled).

11. A method for treating intestinal inflammatory processes comprising the step of:
perorally or rectally administering an effective amount of a lipopolysaccharide.

12. The method of claim 11 wherein the lipopolysaccharide is an agonistic lipopolysaccharide.

13. The method of claim 11 wherein the lipopolysaccharide is a lipopolysaccharide from E. coli.

14. The method of claim 11 wherein the lipopolysaccharide is a lipopolysaccharide from E. coli serotype O2:K1:H6.

15. The method of claim 11 wherein the lipopolysaccharide is a lipopolysaccharide from E. coli serotype O2:K1:H6, strain Laves 1931.

16. The method of claim 11 wherein the effective amount of a lipopolysaccharide is in the range of 0.05 ng/kg/d to about 100 μg/kg/d.

17. The method of claim 11 wherein the effective amount of a lipopolysaccharide is in an oral dosage range of 1 ng/kg/d to 10 μg/kg/d.

18. The method of claim 11 wherein the intestinal inflammatory processes are produced as responses to infection.

19. The method of claim 11 wherein the intestinal inflammatory processes are produced by noxious agents.

20. The method of claim 11 wherein the intestinal inflammatory processes are produced by chemical noxae.

21. The method of claim 11 wherein the intestinal inflammatory processes results from autoimmune diseases.

22. The method of claim 11 wherein the intestinal inflammatory processes comprises colitides or allergic reactions.
